(19) United States
(12) Patent Application Publication
(10) Pub. No.: US 2025/0209346 A1
(43) Pub. Date: Jun. 26, 2025

(54) SYSTEM AND METHOD OF ARTIFICIAL INTELLIGENT (AI) PLATFORM FOR SELECTIONS OF CLINICAL DEVICES AND/OR PRODUCTS BASED ON THE JOB FUNCTION OF END USER OR INTENDED CLINICAL PROCEDURE

(71) Applicant: Viskoot LLC, McLean, VA (US)

(72) Inventors: HAMID REZA SHAFIE, McLean, VA (US); HAOBO CHENG, Greenbelt, MD (US); RUITAO ZHANG, Arlington, VA (US); DEBRUP BASU, Arlington, VA (US)

(21) Appl. No.: 18/983,583

(22) Filed: Dec. 17, 2024

Related U.S. Application Data
(60) Provisional application No. 63/613,782, filed on Dec. 22, 2023.

(51) Int. Cl. G06N 5/01; G16H 40/40

(52) U.S. Cl. CPC G06N 5/01; G16H 40/40

(57) ABSTRACT

The system configured to deliver AI-generated recommendations and guidance is a medical device/surgical products recommendation and guidance AI tool for device selection, application method and clinical indications based on user's job function and preferences. The system includes a training component that improves its ability to recommend new medical devices/surgical products, their applications, and indications to use them. The system is designed to provide users with tailored suggestions and guidance in the realm of clinical applications. Additionally, it enables end-users to identify the spare parts for their medical devices. Its core functionality revolves around analyzing user preferences to offer recommendations for medical devices and surgical products. This automated learning process can be encoded in a training module, and it can improve over time, identifying commonly used control code, visualizations, and configurations for clinical product/device selection across various clinical disciplines.

### CROSS-REFERENCE TO RELATED APPLICATION

This application claims the benefit under 35 U.S.C. § 119 (c) of provisional patent application No. 63/613,782, filed Dec. 22, 2023, the contents of which are hereby incorporated by reference.

### BACKGROUND

It is important to recognize major changes in the demographics of active surgeons, medical/dental/veterinary providers, and their behavior when making decisions to purchase surgical/medical dental/veterinary products. Baby boomer clinicians and office staff are retiring at a rapid pace and most of the active clinicians are Generation X who are digitally immigrant and millennials followed by Gen-Z, who are digitally native. Most of them demand the convenience of searching product information online leading to a convenient purchase from their smartphones or PC.

For decades medical/dental/veterinary manufacturers have established relationships with dealers and distributors as an efficient and effective way to sell and maintain their offerings. Over the last 20 years, “classic” distributor/dealer business models are giving way to technology platforms that deliver value and information “directly” to clinicians and office staff. These platforms provide the clinicians and office staff with improved services across the decision making spectrum, from general education and awareness, through comparative analysis and selection, through purchasing and delivery/application, to maintenance and repair.

Every B2B industry must realize that the changes in B2C have also impacted B2B procurement. Today's B2B buyers such as private clinical offices expect the same customer experience researching and purchasing products in their offices as they do surf the web for personal products. What buyers want is a self-serve model, where they can purchase what they want, when they want, and where they want.

The competitive landscape for medical devices in the next decade is poised to look completely different than it does today, thanks to new and non-traditional entrants, disruptive technologies, and players with global ambitions emerging from high-growth markets. New digital technologies have led to offering of higher quality of care at lower costs driving the healthcare industry toward digital transformation. Some E-commerce companies have already entered the market, leveraging its vast logistics capabilities and huge customer base, with some companies featuring a vast selection of medical supplies like infusion pumps, catheters, IV bags, sutures, forceps, hospital beds, scalpels, and other lab items. They could undercut margins by as much as percent, putting pressure on established medical device distributors and manufacturers. Over time, it is expected that these new entrants will overcome regulatory barriers and move upmarket to sell higher-end products. Additionally, some of these businesses are partnering with life sciences and genomics companies and hiring their healthcare experts, signaling their intent to create new value propositions for patients and consumers.

### SUMMARY

The artificial intelligent (AI) system and method of the disclosed invention with its powerful tools improves the clinicians' and office staff's experience by providing instant access to relevant information online. They can have real-time interaction with the system of the disclosed invention as virtual product specialists. After obtaining all relevant information, the AI system of the disclosed invention is capable of connecting the clinicians or office staff to an e-commerce platform to purchase the desirable medical/dental/veterinary device or product.

The AI system of the disclosed invention delivers quality and up-to-date information that is generally, available to clinicians. At the end, patients win by receive a better care from a better informed clinician. In the healthcare industry, being timely is often the key since it's hard to predict when a clinicians need clinician and technical advice and when a private practice or hospital will need surgical equipment. Traditional dealers operate nine to five and there is no chance to reach a sales rep after hours. With the AI system of the disclosed invention, private offices and hospital can procure what a doctor need 24/7.

The AI system of the disclosed invention is significantly more knowledgeable compared to an individual products consultant with limited knowledge. The AI system narrows product specialist role by minimizing manufacturer's dependency on a particular individual ability to learn, remember and communicate relevant information to clinicians and office staff. The AI system May lead to a significant payroll cost saving for any medical/dental and veterinary device/product manufacturer, and solve the problem of inaccuracy and inconsistency of technical information provided to clinicians and office staff by different product representatives.

These days, every clinical and office staff will start his/her research online. The AI system of the disclosed invention uses digital technology and techniques to engage clinicians and office staff earlier and deliver relevant information about the medical/dental/veterinary devices and products in real-time to foster better decision-making about applications and know-how about the best use of a particular product.

In today's digital age, the AI system's technology and its initiative to establish a “digital relationship” with clinicians and office staff, then remain engaged throughout their decision cycle. The “digital relationship” allows the AI system to interact with surgeons and office staff during their decision-making cycle, and throughout the purchase and post-purchase process much more frequently than any traditional medical/dental veterinary dealer would ever be allowed to interact or could ever afford to maintain with door-to-door sales representatives.

These advantages and others are achieved, for example, by an artificial intelligent (AI) system configured to deliver AI-generated recommendations and guidance for selections of clinical devices and/or products. The AI system includes a guidance server coupled to one or more content providers and one or more system databases. The guidance server includes at least one non-transitory storage medium to store executable instructions and at least one processor to execute the executable instructions that cause the at least one processor to perform operations. The operations include steps of receiving a user input including a job function, preferences and requirements, receiving a dataset corresponding to the job function from the one or more system database, performing machine learning (ML) training by using the dataset with diverse ML models, evaluating and/or testing the trained ML models by using the dataset, generating a prediction of recommended products and/or devices, based on the best ML model, performing matching process of the recommended products and/or devices with products/devices contents in the content provider, and outputting the information of the matched products/devices contents of the content provider. The dataset includes lists of devices and products with features and manufactures information. The evaluating and/or testing the trained ML models includes selecting the best ML model that demonstrates superior performance among the ML models.

These advantages and others are also achieved, for example, by a method to deliver artificial intelligent (AI)-generated recommendations and guidance for selections of clinical devices and/or products via either (i) an AI system including a guidance server or (ii) an integrated development environment (IDE) system in a cloud system. The method includes steps of receiving a user input including a job function, preferences and requirements, receiving a dataset corresponding to the job function from one or more system database, performing machine learning (ML) training by using the dataset with diverse ML models, evaluating and/or testing the trained ML models by using the dataset, generating a prediction of recommended products and/or devices, based on the best ML model, performing matching process of the recommended products and/or devices with products/devices contents in a content provider, and outputting the information of the matched products/devices contents of the content provider. The dataset includes lists of devices and products with features and manufactures information. The evaluating and/or testing the trained ML models includes selecting the best ML model that demonstrates superior performance among the ML models.

### BRIEF DESCRIPTION OF THE DRAWINGS

The preferred embodiments described herein and illustrated by the drawings hereinafter are to illustrate and not to limit the invention, where like designations denote like elements.

FIG. 1 is a diagram illustrating an artificial intelligent (AI) system of the disclosed invention configured to provide a solution for selections of medical, dental, and/or veterinary devices and/or products and clinical application recommendations.

FIG. 2A is a flowchart illustrating an outline of a method of the disclosed invention to provide a solution for selections of medical, dental, and/or veterinary devices and/or products and clinical application recommendations.

FIG. 2B is a flowchart illustrating processes of detecting AI content through comparison with training data.

FIG. 3 is a diagram of components or modules of the AI system. of the disclosed invention.

FIG. 4 is a diagram illustrating a process flow to identify job function that is conducted through user input architecture.

FIG. 5A is a diagram illustrating a process flow for training of one or more machine learning (ML) models that is used in the backend architecture.

FIG. 5B is a process of the step 503 shown in FIG. 5A in which the dataset is divided into training and testing datasets.

FIG. 5C is a process of the stratified sampling that is used in the step 522 shown in FIG. 5B.

FIG. 6 is a diagram illustrating a process flow of backend architecture.

FIG. 7 is a diagram of an exemplary specific process flow for a general practitioner shown in FIG. 6.

FIG. 8 is a diagram of a process flow of the frontend architecture.

FIG. 9 is a process flow for cloud architecture incorporating integrated development environment (IDE) system.

FIG. 10 is a process flow for network architecture for end-users.

FIG. 11 is a process flow for application programming interface (API) connection.

FIG. 12 is a process flow for connecting the system to other medical/surgical device websites.

FIG. 13 is a process flow for image detection feature.

FIG. 14 is a process flow for mobile app feature.

FIG. 15 is an exemplary decision tree for wisdom tooth surgery.

FIG. 16 shows exemplary products that may be recommended by the AI system of the disclosed invention based on specific clinical procedures for wisdom tooth surgery.

FIG. 17 is an exemplary decision tree for vertical ridge augmentation.

FIG. 18 shows exemplary products that may be recommended by the AI system of the disclosed invention based on specific clinical procedures for Allograft Bones.

### DETAILED DESCRIPTION

The following detailed description is merely exemplary in nature and is not intended to limit the described embodiments or the application and uses of the described embodiments. All of the implementations described below are exemplary implementations provided to enable persons skilled in the art to make or use the embodiments of the disclosure and are not intended to limit the scope of the disclosure, which is defined by the claims. It is also to be understood that the drawings included herewith only provide diagrammatic representations of the presently preferred structures of the present invention and that structures falling within the scope of the present invention may include structures different than those shown in the drawings.

The AI system and method of the disclosed invention provide a solution within the domain of medical, dental, and/or veterinary devices and/or products' selection and clinical application recommendations. In addition to proffering well-suited products in accordance with user preferences, the system distinguishes itself by imparting valuable guidance and educational insights. This intelligent framework not only provides recommendations that align with specific user preferences for medical/dental/veterinary devices/products but also enriches these suggestions with supplementary educational information. Through the delivery of comprehensive details, the system of the disclosed invention empowers users to make informed and judicious selections of products and application technique that are precisely tailored to their individual needs and preferences. The system and method of the disclosed invention provide the following features.

### Artificial Intelligent (AI) Functionalities:

The AI system of the disclosed invention is a cutting-edge medical device and surgical product recommendation tool. It provides guidance on device selection, application methods, and clinical indications. The AI incorporates a training component for continuous improvement in recommending new products. It analyzes user preferences, machine learning algorithms, and aggregated data from users, manufacturers, publications, and developers. The system introduces a unique feature categorizing users into General Practitioners, Specialty Clinicians, and Office Staff based on job functions. It tailors recommendations and information based on the user's job function, offering relevant medical equipment or products. The backend architecture utilizes machine learning models to process user inputs and generate predictions for recommended products based on types of treatments. The frontend displays personalized recommendations to users, enhancing the decision-making process in the medical field.

### User Categorization and Personalization:

The AI system categorizes users into General Practitioners, Specialty Clinicians, and Office Staff based on job functions. The AI hyper-personalizes product categories based on the user's job function, ensuring precise recommendations. It accesses job function-specific datasets for refined user inputs and relevant recommendations.

### Process Flow:

The AI system follows a general process flow involving user input collection, backend processing, and frontend processing including display. User inputs, including preferences and requirements, are collected and fed into the backend architecture. The backend, using machine learning algorithms, generates predictions based on the analyzed user inputs. The frontend processing displays the results, allowing users to view and interact with recommended products. This streamlined process ensures a seamless experience from user input to personalized recommendations.

### Training and Model Selection:

Users input their job function, narrowing down the dataset to specific job function-related information. The system undergoes a training phase, utilizing diverse machine learning models to refine predictions. Model evaluation using key performance indicators selects the best-performing model for output generation. The chosen model processes user inputs as a test set, generating predictions for a fine-tuned outcome. The final output is transmitted to the frontend, providing users with a personalized recommendation experience.

### Cloud Architecture:

Cloud architecture involves users providing inputs that enter the cloud system. Inside the cloud, the system accesses aggregated data from the medical device Company website. The Integrated Development Environment (IDE) processes inputs, initiating training analysis on the aggregated data. Training involves control code, automated object mappings, and visualizations. Generated output is recorded and stored within the cloud, later displayed on the user interface for interaction.

### Application Programing Interface (API) Integration:

This integration involves connecting to the medical device website/E-commerce website API for product matching, enhancing the recommendation process. The API integration ensures accurate and efficient product recommendations based on user inputs. The frontend communicates with the API to match backend predictions with the products file. Detailed information about the corresponding product is displayed to users. The frontend provides additional suggestions and educational information for a comprehensive user experience. Users can directly purchase recommended products, streamlining the entire process.

### User Input Processing:

The system processes user inputs through its backend architecture, incorporating machine learning models. The system hyper-personalizes recommendations based on the user's job function, enhancing relevance. The user input collection, training, model selection, and output generation ensure accuracy and personalization.

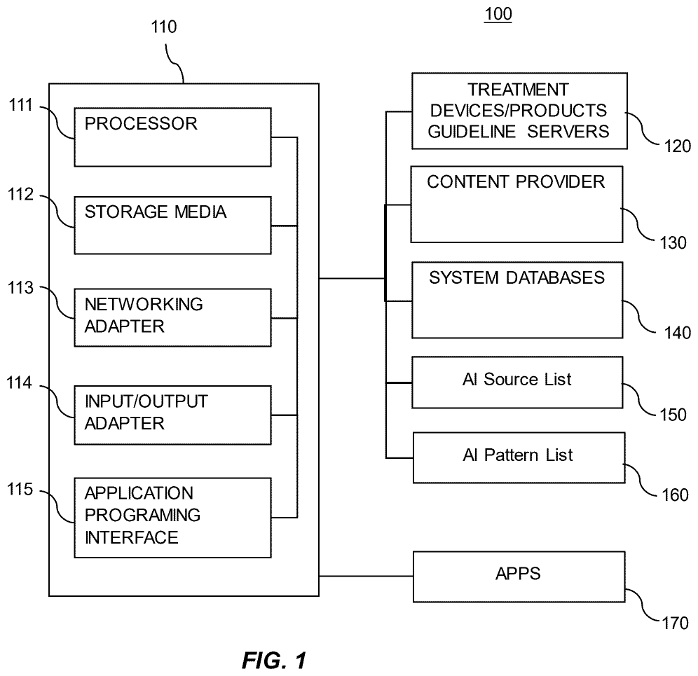

With reference to FIG. 1, shown is a diagram illustrating an artificial intelligent (AI) system 100 of the disclosed invention that utilizes machine learning (ML) technique and is configured to provide a solution for selections of medical, dental, and/or veterinary devices and/or products and clinical application recommendations. The AI system or platform 100 includes a guidance server 110 configured to perform ML processes and to deliver AI-generated recommendations and guidance. The guidance server 110 is adaptable to serve an app or a web platform accessible on smartphones, tablets, laptops, or desktops of end-users. The guidance server 110 includes at least one processor 111, one or more non-transitory computer-readable storage media 112 such as hard drives, random access memories (RAM) and read only memories (ROM), networking adapter 113 to communicate with external devices, external servers and cloud services through wired or wireless communication means, input/output adapter 114 that provides one or more user interfaces, such as displays, keyboards, mouse/mice, etc., to interact with users, and application programing interface (API) 115 for connection to of external platforms' APIs such as Medical Device Website/E-Commerce Website API for product matching, enhancing the recommendation process.

The AI system 100 of the disclosed invention further includes treatment device/product guideline server 120, content provider 130, system database 140, and artificial intelligence (AI) source 150. The guidance server 110 is configured to receive content generated by one or more content providers 130, which may include a source of data, a source of videos, a source of texts, or a source of audios, to determine that the content is generated by an artificial intelligence source, and to generate an output to change how the content is displayed in a content viewer. The guidance server 110 is also configured to access the AI source list 150 comprising a listing of products and clinical applications sources to determine the best match for the searcher query, to access an AI pattern list 160 including a listing of patterns determined to indicate the best match to determine that the pattern of the content matches at least one of the patterns matches the searcher query, and to generate an output to indicate a variety of options that match the researcher query. The AI source list 150 and the AI pattern list 160 may be incorporated in the guidance server 110.

The guidance server 110 is capable of receiving live data streams that include parameters such as natural language input from office staff or clinicians, clinical application data, and the name of the product or product category. The guidance server 110 facilitates support staff personnel with AI-derived data for recursive medical/dental/veterinary products. The guidance server 110 receives parameters such as natural language input, product image, and clinical application data. The guidance server 110 may be connected to treatment device/product guideline servers 120 that offers different treatment devices and/or product recommendations. The guidance server 110 is configured to deliver AI-generated product recommendations to one or more apps 170. The apps 170 may be accessible on smartphones, tablets, or web applications on laptops or desktops within clinical facilities or on private devices of the searcher.

The guidance server 110 may integrate the product and clinical application recognition processor into the guidance server. When determining the query's nature regarding whether it pertains to a medical, dental and/or veterinary device and/or product and its manufacturer, the guidance server 110 may accesses one or more system databases 140 that include lists of devices and products with features and manufacturers information, making the best match and generating recommendations.

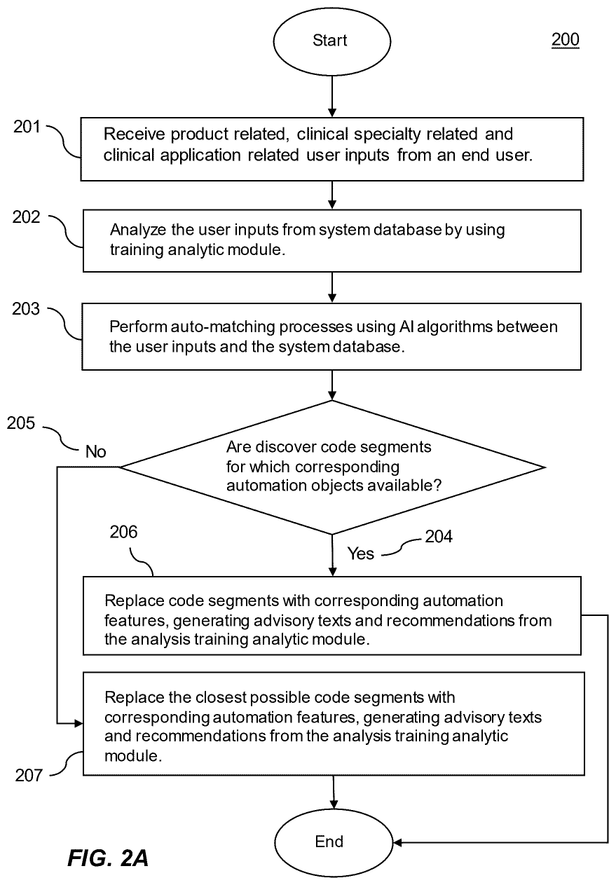

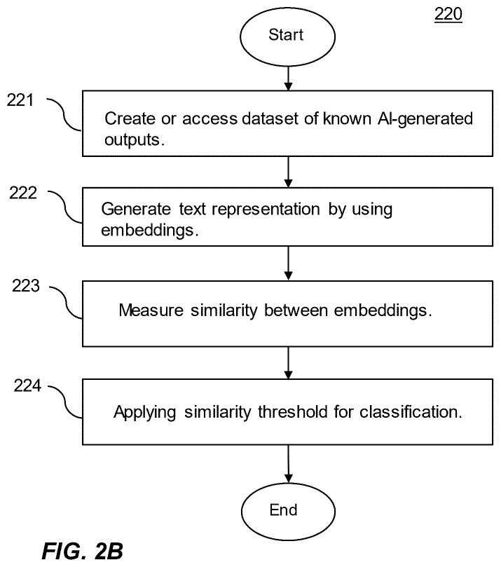

With reference to FIG. 2A, shown is a flowchart illustrating an outline of method 200 of the disclosed invention to provide a solution for selections of medical, dental, and/or veterinary devices and/or products and clinical application recommendations. The one or more computer-readable storage media 112 include instructions that cause the processor 111 to perform the steps and features of computer-implemented method 200.

Referring to FIG. 2A, the method 200 of the disclosed invention includes the following steps. Product related, clinical specialty related, and clinical application related user inputs are received from an end user, block 201. The user inputs are analyzed from the system database 140 by using a training analytic module, block 202. Auto-match processes between the user input and the system database 140 is performed by using AI algorithms via the guidance server 110, block 203. The guidance server 110 may access the AI source 150 to perform the auto-match processes. If discover code segments for which corresponding automation objects are available, block 204, code segments are replaced with corresponding automation features, generating advisory texts and recommendations from the analysis training analytic module, block 206. If discover code segments for which corresponding automation objects are not available, block 205, the closest possible code segments are replaced with corresponding automation features, generating advisory texts and recommendations from the analysis training analytic module, block 207.

The computer-implemented method 200 involves receiving content generated by clinical or support staff searchers. The method includes processes that identifies whether the query is from a clinician (general practitioner, specialist) or support staff. It generates an output that influences the decision-making process of the searcher regarding product selection or application. The method may utilize one or more content providers 130 that include Internet websites. The contents provided by the content providers 130 may include a source of data, a source of videos, a source of texts, or a source of audios. The content viewer may include an Internet browser. The content viewer may include a word processing program or an application executed by an Internet browser. The method includes generating an output to indicate a percentage of the content that is artificial intelligence content.

The step 202 of the method 200 may include steps of receiving content generated by the guidance server 110 or the content provider 130, determining the relevant content according to the job function or clinical procedure, and generating an output to change how the content is displayed in a content viewer, The step of determining the relevance includes steps of accessing name(s), picture(s), and product description and clinical applications according to the job function or intended clinical use, determining the best product option, comparing the best-recommended option against the alternative product option list, and guiding the searcher to choose the desired option and proceed to the e-commerce platform for checkout.

The step 202 of the method 200 may further include a process that specifies one or more sources that include a uniform resource locator, an organization, or an internet protocol address. In order to clarify the processes, the method provides processes that include accessing an artificial intelligence pattern list comprising a listing of products related to each job function and clinical application(s) source, analyzing the content to determine a pattern, comparing the searcher query against the artificial intelligence pattern list, and determining that the pattern of the content matches at least one of the patterns determined to be the best option for the searcher. The contents provided by the content providers may be displayed by using a content viewer including an Internet browser. The content viewer may include a word processing program or application executed by an Internet browser.

With reference to FIG. 2B, shown is a flowchart illustrating processes 220 of detecting AI content through comparison with training data. The method 200 may additionally incorporate processes 220 to generate an output to indicate a percentage of the content that is artificial intelligence content. The system 100 can identify whether content is AI-generated by comparing it against a dataset of known AI-generated outputs through the processes 220. This process leverages advanced machine learning techniques, such as cosine similarity or embedding-based approaches, to analyze the text and identify patterns that match those found in AI-generated content.

The first step is creating or accessing a comprehensive dataset of AI-generated outputs, block 221. This dataset may include (i) text generated by AI models like GPT, BERT, etc. or other generative models, (ii) AI-written content from specific platforms or tools, and (iii) examples of typical AI outputs across various domains and styles (e.g., customer support emails, product descriptions, or generic writing). The dataset acts as a benchmark for detecting AI-generated content. It may include diverse examples to account for the variability in AI-generated text. Text representation is generated by using embeddings, block 222. To compare content, the system 100 transforms both the input text and the dataset examples into numerical representations called embeddings. Pre-trained models, such as BERT (Bidirectional Encoder Representations from Transformers) or GPT (Generative Pre-trained Transformer), are commonly used for this purpose. These models convert sentences or documents into high-dimensional vectors, and capture semantic meaning and relationships between words and phrases. Similarity between embeddings is measured, block 223. After generating embeddings for both the input text and the known AI-generated examples, the system measures the similarity between these embeddings. This can be done using cosine similarity, which calculates the cosine of the angle between two vectors in a high-dimensional space. In the cosine similarity, a similarity score close to one (1) indicates high resemblance between the input text and the AI-generated content, and a similarity score closer to zero (0) indicates little to no resemblance. A similarity threshold for classification is applied, block 224. The system 100 applies a similarity threshold to classify the input content. If the similarity score exceeds the threshold, the content is flagged as AI-generated. If the score is below the threshold, it is classified as non-AI content. This threshold can be fine-tuned based on the desired accuracy and the characteristics of the dataset.

In addition to the process 220, advanced features may be further implemented in the process 220 to improve detection accuracy. The system 100 may incorporate additional features: for example, (i) Contextual Analysis: examining how the content aligns with typical usage scenarios for AI, such as repetitive structures, fixed templates, or lack of contextual depth, (ii) Stylistic Patterns: identifying specific writing styles associated with AI models, such as overuse of certain connectors (“therefore,” “however,” etc.) or predictable sentence rhythms, and (iii) Metadata: checking for embedded AI markers or generation flags that some AI systems may include.

The process 220 provides benefits of, for example, (i) accuracy: comparing embeddings allows for precise identification of AI-generated content, especially when using robust pre-trained models, (ii) scalability: embedding-based approaches are computationally efficient and can process large datasets quickly, and (iii) adaptability: the system can update its dataset of known AI-generated outputs to keep pace with evolving AI technologies. By leveraging pre-trained models and similarity-based methods, the system can efficiently and accurately distinguish AI-generated content from human-written content. This approach provides a scalable and adaptable solution for identifying AI content across various domains.

For a working example of the process 220, let's consider a scenario where a system analyzes a product description to detect AI content.
Input text: “This revolutionary gadget offers unparalleled performance and reliability, thanks to cutting-edge technology.”
Dataset example: AI-generated product descriptions from a generative model like GPT-3.
Process follows the steps:
   Step 1: The input text is converted into an embedding using BERT.
   Step 2: The AI-generated examples in the dataset are also converted into embeddings.
   Step 3: The cosine similarity score between the input embedding and the dataset embeddings is calculated.
   Step 4: If the similarity score is 0.95 (high resemblance), the system flags the input as AI-generated.

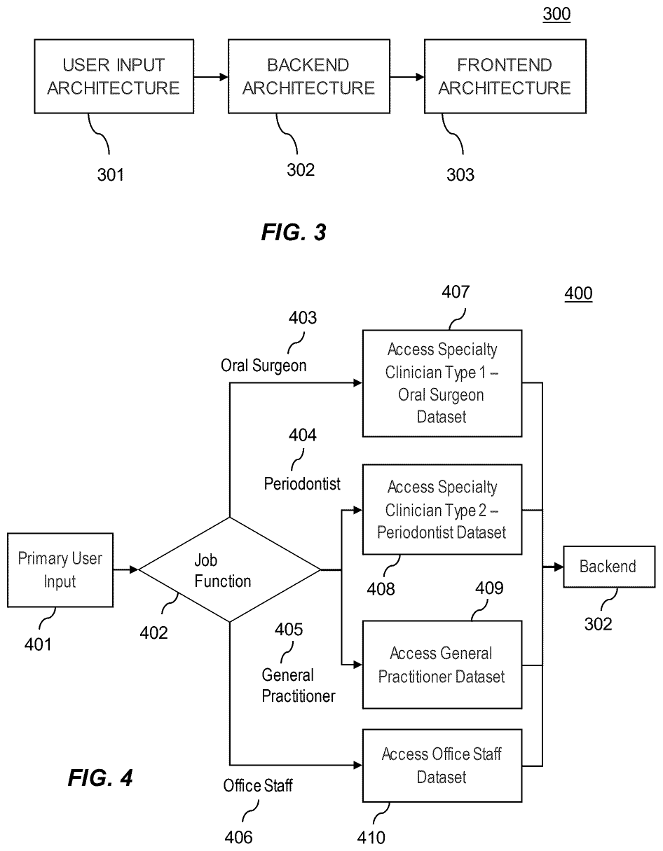

With reference to FIG. 3, shown is a diagram of components or modules 300 of the AI system 100, which are programing codes stored in the storage media 112 and include instructions that cause the processor 111 to perform operations to provide a solution for selections of medical, dental, and/or veterinary devices and/or products and clinical application recommendations. The components 300 include user input architecture 301, backend architecture 302, and frontend architecture 303. The method of the disclosed invention follows the general process flow of the components 300 involving processes in user input architecture 301 and their subsequent processing through the backend architecture 302, which incorporates machine learning (ML) algorithms, and processing through the frontend architecture 303. The storage medium 112 of the guidance server 110 stores program codes for the processing through the user input architecture 301, the backend architecture 302, and the frontend architecture 303.

In the user input architecture 301, users provide inputs, which include preferences, requirements, or any relevant information related to surgical product selection. The user inputs are collected through the processing of the input architecture 301, and the collected user inputs are fed into the backend architecture 302. The backend architecture 302 employs ML algorithms to analyze and process the input data. The backend architecture 302 generates predictions for recommended products, based on the machine learning algorithms' analysis of user inputs. The results of the predictions are conveyed to the frontend architecture 303. The frontend architecture includes user interfaces that users interact with, and displays the recommended products generated by the backend architecture 302. Users may view the recommended products generated in the frontend architecture 303. This general process flow through the components 300 encapsulates the movement of information from the user to the backend architecture 302, where machine learning algorithms process the data to make predictions. The results are then presented to the user on the frontend 303 for a seamless and user-friendly experience. The user input architecture 301 is also configured to identify and categorize users according to their specific job functions.

With reference to FIG. 4, shown is a process flow to identify job function 500 that is conducted through user input architecture 301. The AI system and method of the disclosed invention introduce a distinctive feature designed to tailor user experiences by identifying and categorizing users according to their specific job functions. The users may be categorized in groups including General Practitioners, Specialty Clinicians, and Office Staff. General practitioner is an individual who has completed medical school, or dental school, or veterinary school but hasn't pursued specialized training after graduation. Specialist clinical is an individual who has pursued a specialty training by enrolling in a residency program after graduation from medical school or dental school or veterinary school. Office staff is an individual who can be clinical assistant or administrative personnel who is supporting the practicing clinician.

For description purpose, FIG. 4 exemplarily shows dentistry as a medical field to demonstrate the functionality of the AI system 100 of the disclosed invention when general dentists, specialist dentists (oral surgeons and periodontists), and/or office staff are interacting with the AI system 100. In this example, a user may choose dental field as a user input, and then the user can select his/her job function from one of those options of general dentists, spiciest dentist and office staff, block 401. The AI system 100 utilizes this user-selected job function 402 to hyper-personalize product categories, ensuring that recommendations are finely tuned to the specific needs of each professional role. Furthermore, it accesses job function-specific datasets to fetch relevant user inputs, enhancing the precision and relevance of its recommendations. The job function-specific dataset may be stored in the system database 140.

As shown in FIG. 4, if the job function 402 is Oral Surgeon 403, the guidance server 110 accesses or utilizes datasets for specialty clinician Type 1 (oral surgeon), block 407. If the job function 402 is Periodontist 404, the guidance server 110 accesses or utilizes datasets for specialty clinician Type 2 (periodontist), block 408. If the job function 402 is General Practitioner 405, the guidance server 110 accesses or utilizes datasets for General Practitioner, block 409. If the job function 402 is Office Staff 406, the guidance server 110 accesses or utilizes datasets for office staff, block 410. The selected job function and datasets are used in the processes in the backend architecture 302, such as for training and selecting ML models. The datasets for the job function may be stored in the system database 140.

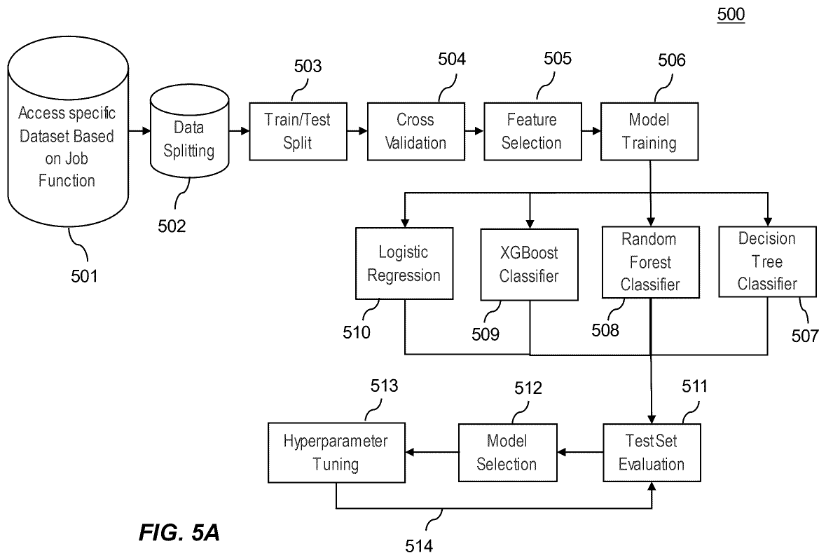

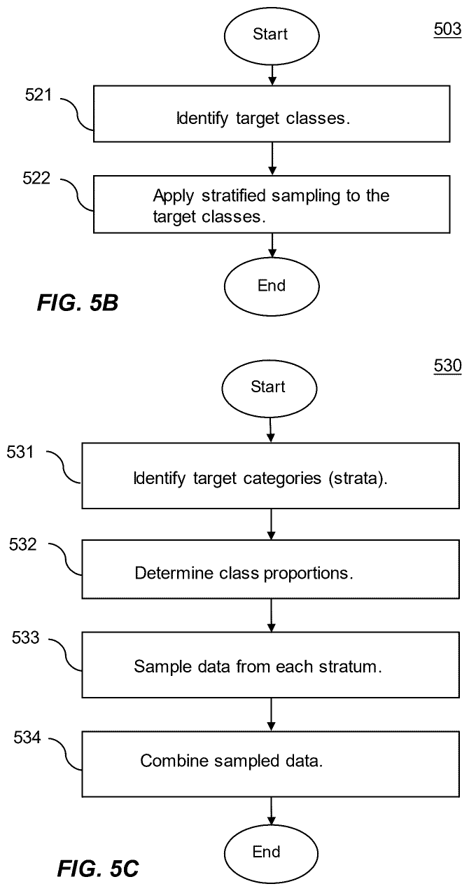

With reference to FIG. 5A, shown is a process flow 500 for training of one or more machine learning (ML) models that is used in the backend architecture 302. When a user interacts with the AI system 100, it starts by accessing the comprehensive system database 140. The user specifies their job function, helping the AI system 100 narrow down the dataset to specific job function-related information, as described referring to FIG. 4. Based on the job function, one of these datasets is selected and focused. The focused dataset ensures that user inputs are tailored to the selected job function and relevant product categories.

The ML training process begins with access to a specific dataset based on job function, block 501. The dataset is split, block 502. Specifically, the dataset is divided into training and testing datasets, block 503, which is described in detail below referring to FIG. 5B. The training dataset facilitates the model training, while the testing dataset serves to evaluate performance. In order to utilize k-fold cross-validation, the dataset is divided into k subsets (where k is an integer), block 504, instead of a single split, and the ML models undergo training and testing k times for robust evaluation. Features are identified and selected, block 505. It is determined which features, such as job function and preferences, would contribute to training the ML models.

The ML models are trained, block 506, with training dataset. The ML model training 506 may include the processes 507-510. In the decision tree classifier process, block 507, decision trees are employed to partition the data based on feature values. Nodes represent decision points, and training involves finding optimal splits for each node. In the random forest classifier process, block 508, an ensemble of the decision trees is created. Each ensemble is trained on random data and features to achieve robust predictions through a voting mechanism. In the extreme gradient boosting (XGBoost) classifier process, block 509, utilizing extreme gradient boosting, a series of weak learners (typically decision trees) is built, and the built series of the weak learners are combined for enhanced predictive power. In the logistic regression process, block 510, regression serves as a classification algorithm. The logistic regression is a statistical method used for binary classification tasks. The goal is to predict the probability of an event occurring based on one or more input variables. Training involves finding coefficients maximizing the likelihood of observed data.

Once the ML models are trained, evaluation of the ML models is conducted, block 511, with testing dataset. The ML models are rigorously evaluated on the test set for an unbiased performance estimate. For the evaluation, common metrics, such as accuracy, precision, recall, F1 score that measures a model's accuracy by combining its precision and recall scores, and area under the receiver operating characteristic (ROC) curve, may be used. Among the trained ML models, best performing model is selected, block 512. After thorough evaluation 511, the model demonstrating superior performance on the test set is selected. Once the best-performing model is identified and selected 512, hyperparameter tuning is conducted, block 513. This process involves cither systematically exploring a predefined set of hyperparameters (Grid Search) or randomly sampling hyperparameters (Random Search). Hyperparameters were fine-tuned using cross-validation, systematically evaluating performance for different hyperparameter combinations. The hyperparameter set delivering optimal performance on the validation set is selected for the final ML model.

After the hyperparameter tuning 513, test set evaluation 511 is conducted with the hyperparameter tuned ML model, block 514. With the tuned hyperparameters, the ML model is rigorously evaluated on the test set to obtain an unbiased estimate of its performance. Through the evaluation, standard metrics, such as accuracy, precision, recall, F1 score, and the area under the ROC curve, guide the analysis of performance of the model. Based on the evaluation results, areas where the model excels are observed, and potential areas for improvement are identified. These processes of evaluation, analysis, and fine-tuning, blocks 511-513, are often iterative till optimized results are achieved, ensuring that each adjustment contributed positively to overall model effectiveness.

With reference to FIG. 5B, shown is a process of the step 503 (shown in FIG. 5A) in which the dataset is divided into training and testing datasets. In this step, Stratified sampling is employed to ensure a balanced and representative division of the dataset. This approach ensures a balanced class distribution across both subsets, particularly for classification problems involving categorical targets such as job functions or preferences. The stratified sampling is a method of dividing a dataset into subsets (strata) based on specific characteristics or categories. Each subset maintains the proportional representation of these characteristics as they appear in the entire dataset. It can be used when the dataset involves categorical variables, such as job functions, user preferences, or any other class labels, especially in classification problems.

Referring to FIG. 5B, the step 503 further includes identifying target classes, block 521. The categorical target variables (e.g., job functions or user preferences) are analyzed to determine their distribution in the overall dataset. Stratified sampling is applied to the identified target classes, block 522. The dataset is split into training and testing subsets while preserving the proportion of each target class in both subsets. This ensures that rare or minority classes are adequately represented. After stratified sampling in the step 522, k-fold cross-validation further divides the dataset into k subsets, rotating the training and testing roles to evaluate the model comprehensively, as described referring to the step 504. Stratified k-fold cross-validation is often used to maintain class balance across folds.

With reference to FIG. 5C, shown is a process of the stratified sampling 530 that is used in the step 522. The target categories (strata) are identified, block 531. The dataset is grouped by a categorical variable (e.g., job functions such as Oral Surgeon, Office Staff, and General Practitioner). Each unique value in this categorical variable forms a stratum. Class proportions are determined, block 532. The proportion of each category in the overall dataset is calculated. For example, if “Oral Surgeon” accounts for 40% of the dataset and “Office Staff” for 20%, these proportions will guide the division into training and testing sets. Data from each stratum is sampled, block 533. For each category, data points are sampled such that their proportions in the training and testing datasets match their proportions in the entire dataset. This ensures that rare classes are not excluded or overrepresented. The sampled data is combined, block 534. The sampled data from each stratum is combined to form the final training and testing subsets.

The stratified sampling provides a benefit of balanced class distribution. By preserving the proportional representation of each class (e.g., job functions such as “oral surgeon,” “periodontist,” etc.), the stratified sampling avoids scenarios where certain classes are underrepresented in either the training or testing datasets. This ensures the model is trained on diverse data and evaluated on a representative test set. The stratified sampling further provides a benefit of improved model generalization. Maintaining the class balance prevents bias toward overrepresented classes, improving the model's ability to generalize to new data. The stratified sampling also provides a benefit of consistency across subsets. With stratified sampling, both the training and testing datasets reflect the overall class distribution, ensuring a fair and consistent evaluation.

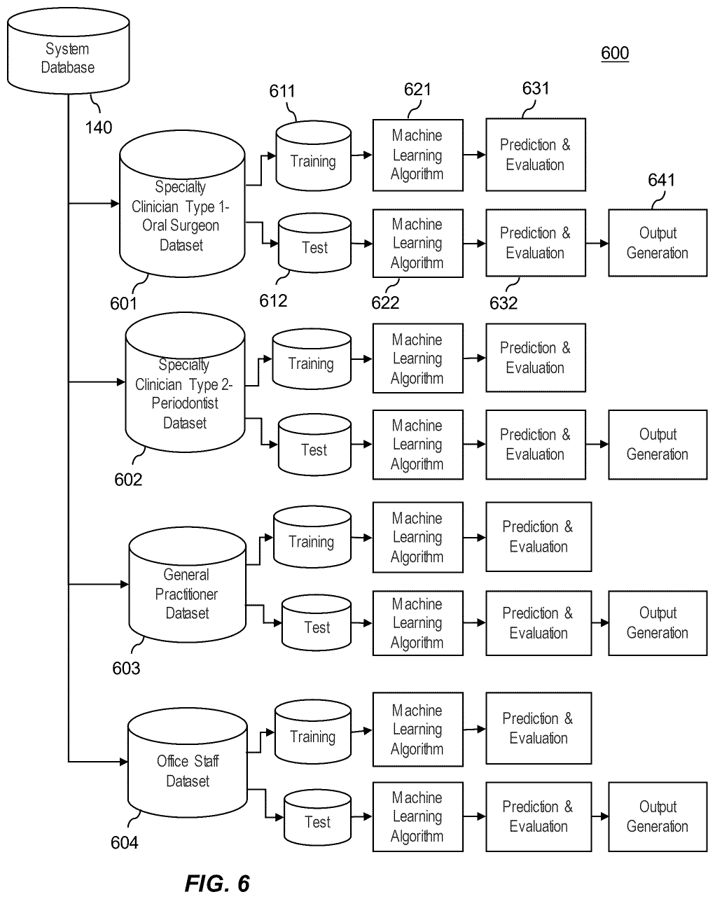

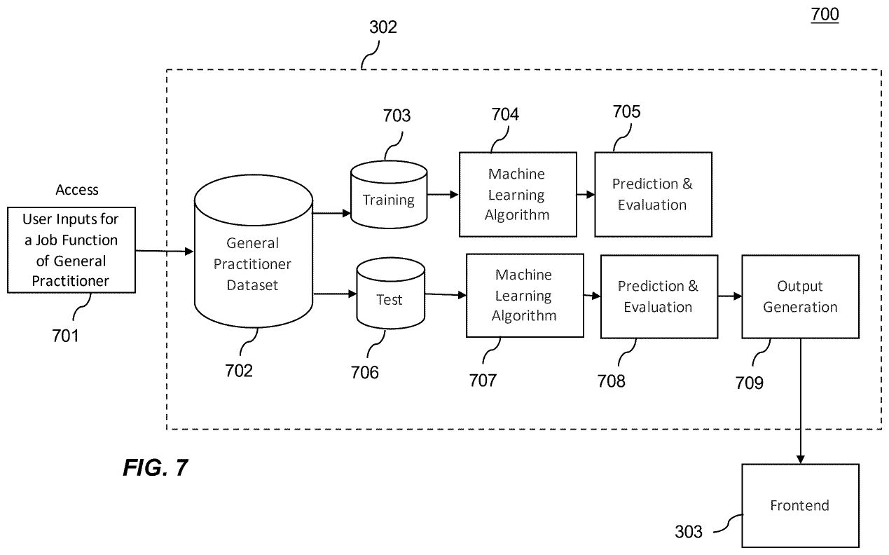

With reference to FIG. 6, shown is a process flow of backend architecture 302. As described referring to FIG. 4, in the example of dentistry, the system database 140 includes a specialty clinician Type 1 (oral surgeon) dataset 601, a specialty clinician Type 2 (periodontist) dataset 602, a general practitioner dataset 603, and an office staff dataset 604. Based on the job function, one of these datasets 601-604 is selected and focused. The focused dataset ensures that user inputs are tailored to the selected job function and relevant product categories.

After the user provides all the necessary inputs, the AI system 100 initiates training 611 using various machine learning (ML) models, as described referring to FIG. 5A. This training process 611 involves teaching the system by using ML algorithms 621 to make predictions based on past data from the selected job function dataset. Once the teaching by using ML algorithms 611 is completed, the system evaluates the predictions 631 made by different ML models using key performance indicators. The best-performing ML model is then selected for further use. These processes are described in detail referring to FIG. 5A. Once the best-performing ML model is selected, test processes 612 are conducted with the model. The user inputs are introduced to this selected model as a test set. By applying ML algorithms 622, predictions 632 are generated based on this refined model. The final output 641, derived from the user inputs and the selected machine learning model, is then sent to the frontend 303 for the user to review. This ensures a personalized and accurate recommendation experience based on the user's specific job function. These processes 611-641 are applied to each of the job function datasets 601-604.

With reference to FIG. 7, shown is an exemplary specific process flow for a general practitioner 700 shown in FIG. 6. The process flow includes the following processes. User Inputs for a job function is received, block 701. In this example, the user selects General Practitioner for the job function. The AI system 100 accesses the system database 140, refining the dataset to align precisely with General Practitioner group 702 and relevant product categories. Upon user input completion, the AI system 100 undergoes a training phase 703. In the training, diverse machine learning (ML) models and algorithms 704 are employed to instruct the AI system 100 based on information and historical data from the General Practitioner dataset. In the post-training process, the AI system 100 evaluates predictions 705 from various models using key performance indicators, and the best performing model is selected for further use, as described referring to FIG. 5A. Once the best-performing ML model is selected, test processes 706 are conducted with the best-performing model. User inputs serve as a test dataset for the selected best-performing model. ML algorithms 707 are applied to the selected best-performing model, and predictions 708 for a fine-tuned outcome are generated. The conclusive output 709, derived from user inputs and the selected model, is transmitted to the frontend architecture 303. Through these processes, users interact with a personalized recommendation experience tailored for General Practitioner.

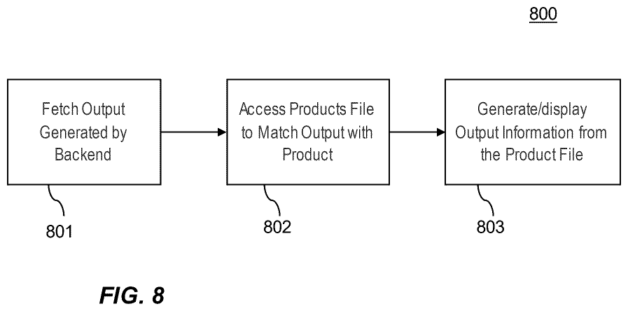

With reference to FIG. 8, shown is a process flow 800 of the frontend architecture 303. Once the backend architecture 302 processes the user inputs and generates a refined output, the frontend architecture 303 interacts with the backend architecture 302 to retrieve the final output, block 801. To enrich the user experience, the frontend architecture 303 communicates with content provider 130, which may include Internet websites, a source of data, a source of videos, a source of texts, or a source of audios, to access product information or files to match the output from the backend 302 with the products in the content provider 130, block 802. For example, the frontend 303 may communicates with API of the medical device website in the content provider 130, such as a products file (presented in JSON format). It searches for a match between the output from the backend architecture 302 and the products file in the content provider 130. When a match is identified, detailed information about the corresponding product is generated as part of the output, block 803. Beyond this, the frontend 303 further provides additional suggestions to the user. It also includes supplementary educational information, ensuring that users not only receive tailored product recommendations but also gain valuable insights related to their specific job function and preferences.

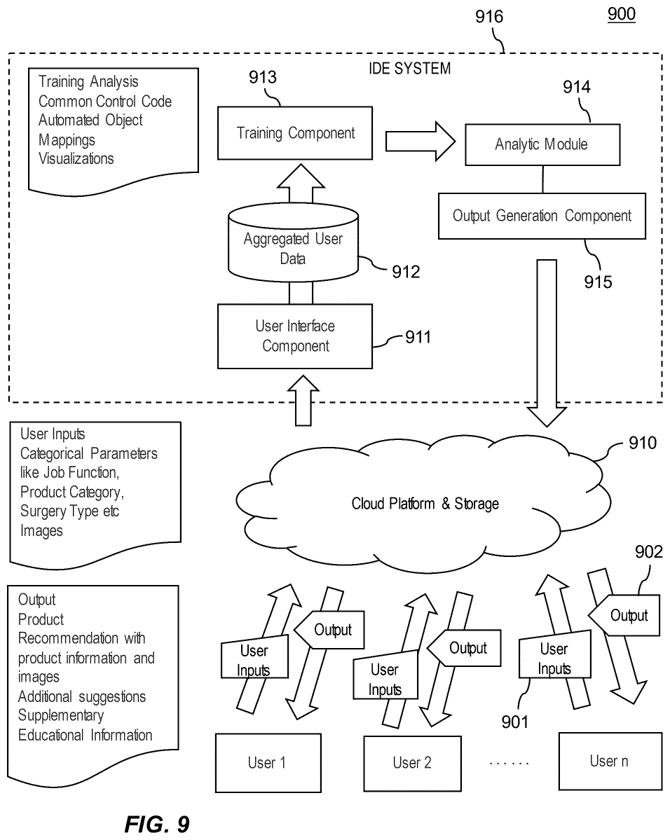

With reference to FIG. 9, shown is a process flow for cloud architecture 900 incorporating integrated development environment (IDE) system. The process flow includes the following processes. Users provide inputs 901 that enter the cloud system 910 that includes IDE system 916. The AI system 100 of the disclosed invention may be incorporated in the IDE system 916 of the cloud system 910. Inside the cloud system 910, the AI system 100 accesses the aggregated database 912 through the user interface 911. In this case, the AI system 100 accesses the data present in the Medical Device Company Website. The integrated IDE system 916 processes inputs, initiating training 913 and analysis 914 on the aggregated data. Training component 913 involves control code, automated object mappings, and visualizations from the aggregated data. Predictions are generated using various machine-learning models during the training component. In the analytic module 914, key performance indicators are employed to analyze results from different machine learning models. The best-performing model is selected for output generation based on user inputs via the output generation component 915. The generated output is recorded and stored within the cloud system 910. The stored output is fed into the user interface (UI) 902. The UI 902 displays the output to the user for interaction and review.

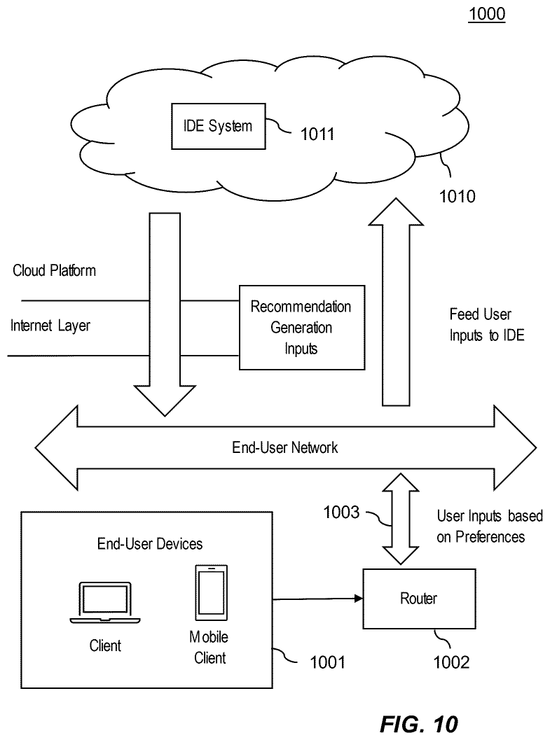

With reference to FIG. 10, shown is a process flow for network architecture 1000 for end users. The process flow includes the following processes. End-users access the AI system 100 through desktop or mobile devices 1001. The users may utilize a router 1002 to establish a connection with the AI system 100. User inputs 1003, reflecting preferences for surgical products, are submitted via the router 1002. Input data is recorded and stored in the cloud 1010. The Integrated Development Environment (IDE) system 1011 processes, trains, and evaluates the input data. Based on the processed data, a product recommendation output is generated via the IDE system 1011. The generated output is sent back to the end-user's device 1001, via the router 1002, for interaction.

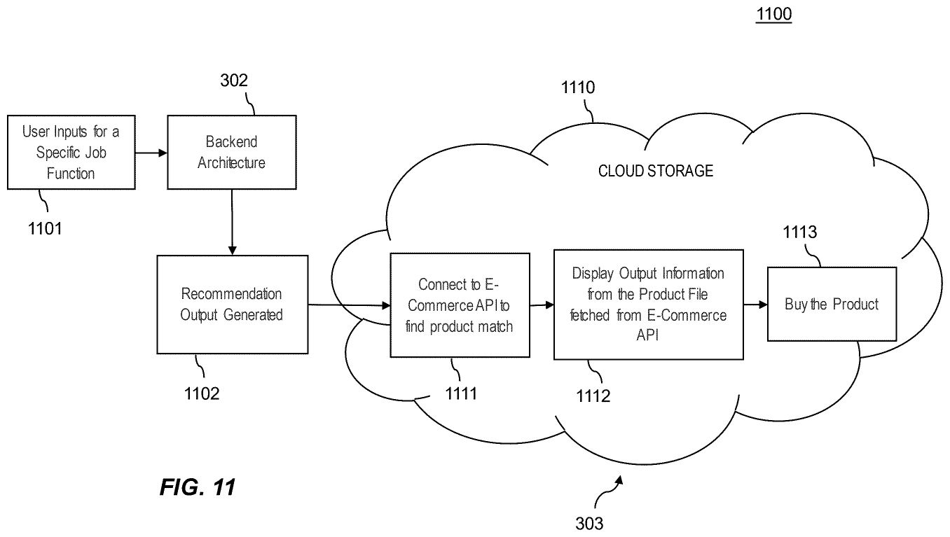

With reference to FIG. 11, shown is a process flow for application programming interface (API) connection 1100. The process flow includes the following processes. Users provide specific job function-related inputs, block 1101. The inputs are sent to the backend architecture 302 for analysis. The backend architecture 302 processes the inputs to generate predictions for recommended product(s). The backend architecture 302 generates a recommendation output based on the analysis and job function, block 1102. The recommendation output triggers a connection to the E-Commerce API, block 1111, in the cloud storage 1110. The API is queried to find a match for the recommended product. Information from the product file fetched from the E-Commerce API is displayed on the frontend, block 1112. The users can view product details and decide to purchase, block 1113. The processes 1111-1112 correspond to the processes of the frontend architecture 303, described referring to FIG. 8. Information related to the frontend processes, including user interactions, is stored in the cloud storage 1110 for future reference.

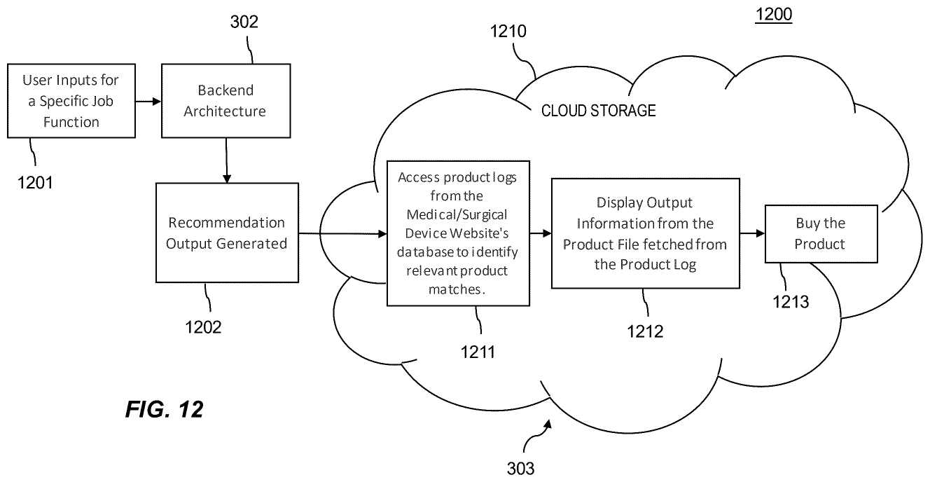

With reference to FIG. 12, shown is a process flow 1200 for connecting the AI system 100 to other medical/surgical device websites. The process flow includes the following processes. Users provide inputs specific to their job function, block 1201. The inputs are sent to the backend architecture 302 for analysis. The backend architecture 302 processes the inputs to generate predictions for recommended product(s). The Backend generates a recommendation output based on the analysis and job function, block 1202. The recommendation triggers access to the Medical/Surgical Device Website's database to identify relevant product matches, block 1211. product logs from the database are analyzed to identify relevant product matches. Information from the product file fetched from the product log is displayed on the frontend, block 1212. The users can view detailed product information and make purchase decisions, block 1213. The processes 1211-1212 correspond to the processes of the frontend architecture 303, described referring to FIG. 8. Information related to the frontend processes, including user interactions and purchases is stored in the cloud storage 1210 for record-keeping.

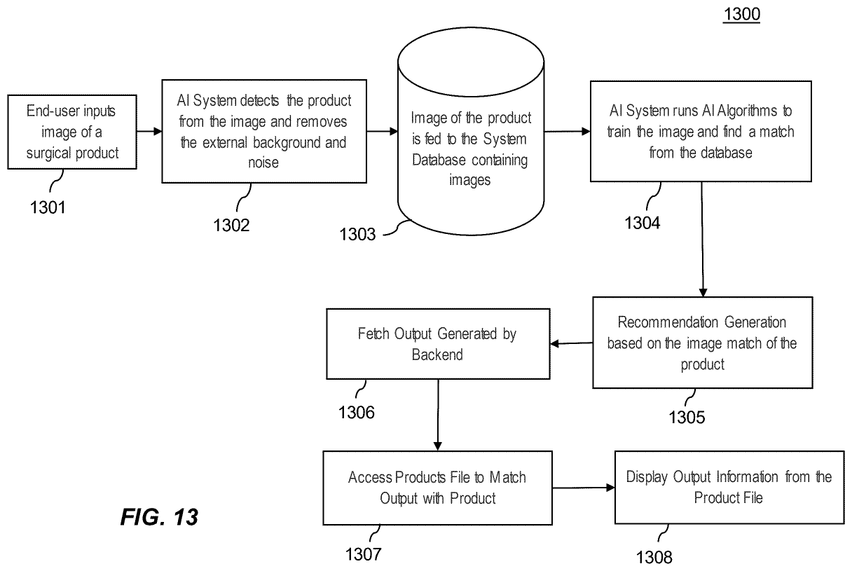

With reference to FIG. 13, shown is a process flow for image detection feature 1300. The process flow includes the following processes. Users upload an image of a surgical product, block 1301. The AI system 100 detects the product from the image, removes external background, and filters out noise, block 1302. The processed product image is fed into the system database containing images, block 1303. The AI system 100 runs AI algorithms to train on the image and identify patterns, block 1304. Trained algorithms compare the input image with images in the system database to find a match. Based on the image match, the AI system 100 generates recommendations for similar products, block 1305. The recommendation output is fetched from the backend architecture 302, block 1306. The AI system 100 accesses the products file to match the recommendation output with detailed product information, block 1307. Detailed information about the recommended product is displayed to the user based on the matched output from the Products File, block 1308.

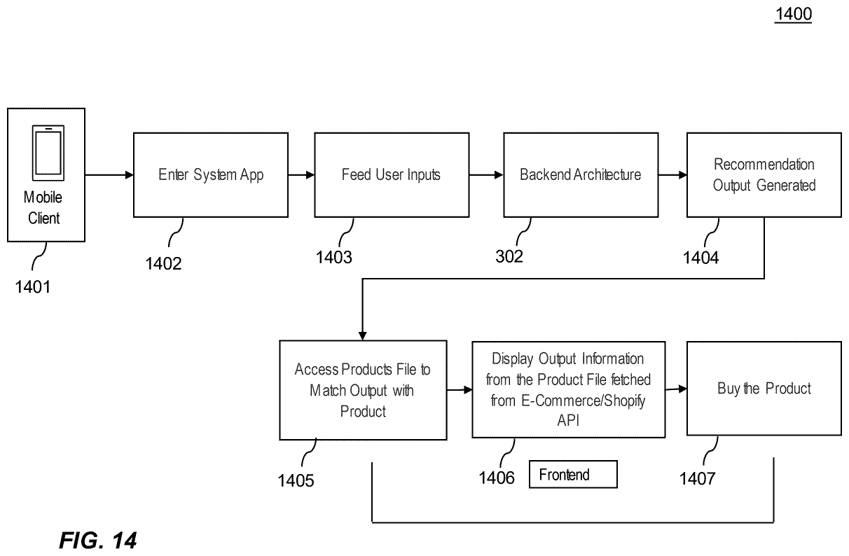

With reference to FIG. 14, shown is a process flow for mobile app feature. The process flow includes the following processes. Users access the AI system application 100 through their mobile devices 1401. Users provide specific inputs related to their job function or product preferences, block 1402. User's inputs are sent to the backend architecture 302 for analysis using machine learning algorithms. The backend architecture 302 generates a recommendation output based on the user's inputs and historical data, block 1404. The AI system 100 accesses the products file to match the recommendation output with detailed product information, block 1405. Detailed information about the recommended product is displayed on the mobile app, block 1406. This information is fetched from the E-Commerce API. Users have the option to buy the recommended product, block 1407, directly through the mobile app, integrating with the E-Commerce platform. The processes 1405-1407 correspond to the processes of the frontend architecture 303, described referring to FIG. 8.

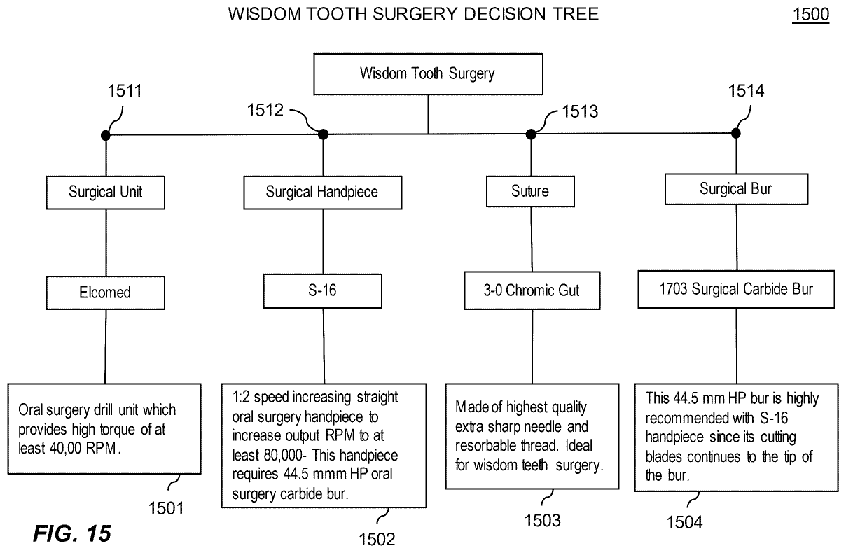

With reference to FIG. 15, shown are a working example of a decision tree 1500 for wisdom tooth surgery. As described referring to step 507 in FIG. 5A, decision trees 1500 are employed to partition the data based on feature values 1501-1504. Nodes 1511-1514 represent decision points, and training involves finding optimal splits for each node. The decision tree 1500 shows exemplary products that may be selected or recommended based on types of treatment for the wisdom tooth surgery.

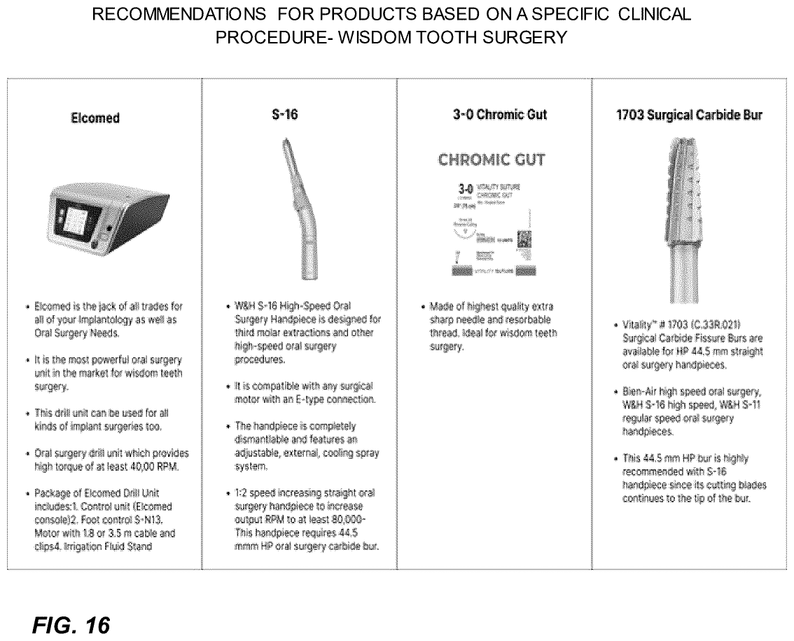

With reference to FIG. 16, shown are exemplary products that may be recommended by the AI system 100 based on specific clinical procedures for wisdom tooth surgery, by utilizing the decision tree 1500 shown in FIG. 15. The products exemplarily include a drill unit (Elcomed), high-speed oral surgery handpiece, high quality extra sharp needle, and surgical carbide bur.

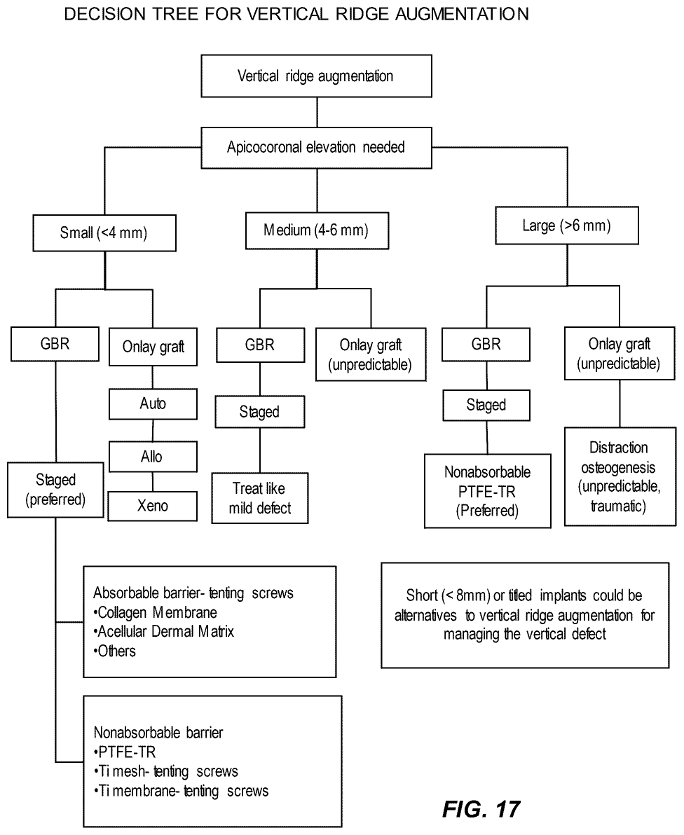

With reference to FIG. 17, shown is a working example of a decision tree for vertical ridge augmentation. The decision tree shows exemplary products that may be selected or recommended based on types of treatment for the vertical ridge augmentation.

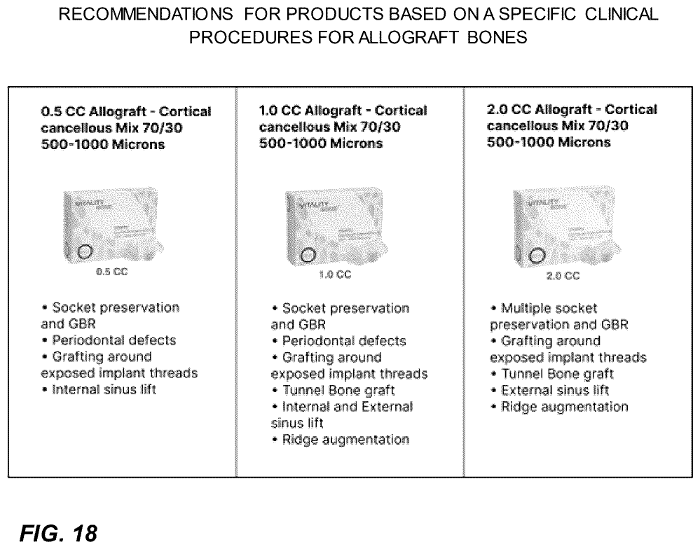

With reference to FIG. 18, shown are exemplary products that may be recommended by the system of the disclosed invention based on specific clinical procedures for Allograft Bones.

Since many modifications, variations, and changes in detail can be made to the described preferred embodiments of the invention, it is intended that all matters in the foregoing description and shown in the accompanying drawings be interpreted as illustrative and not in a limiting sense. Consequently, the scope of the invention should be determined by the appended claims and their legal equivalents.

What is claimed is:

1. An artificial intelligent (AI) system configured to deliver AI-generated recommendations and guidance for selections of clinical devices and/or products, comprising:
a guidance server coupled to one or more content providers and one or more system databases, wherein the guidance server comprises:
at least one non-transitory storage medium to store executable instructions; and
at least one processor to execute the executable instructions that cause the at least one processor to perform operations comprising:
receiving a user input including a job function, preferences and requirements;
receiving a dataset corresponding to the job function from the one or more system database, wherein the dataset comprises lists of devices and products with features and manufactures information;
performing machine learning (ML) training by using the dataset with diverse ML models;
evaluating and/or testing the trained ML models by using the dataset, wherein the evaluating and/or testing the trained ML models comprises selecting the best ML model that demonstrates superior performance among the ML models;
generating a prediction of recommended products and/or devices, based on the best ML model;
performing matching process of the recommended products and/or devices with products/devices contents in the content provider; and
outputting the information of the matched products/devices contents of the content provider.

2. The AI system of claim 1 wherein the operations further comprising splitting the dataset corresponding to the job function into a training dataset and a testing dataset, wherein the ML training is performed by using the training dataset and the testing the trained ML models is conducted with the testing dataset.

3. The AI system of claim 1 wherein the operations further comprising performing k-fold cross-validation in which the dataset is divided into k subsets and the ML models undergo training and testing k times for robust evaluation, where k is an integer.

4. The AI system of claim 1 wherein the operations further comprising identifying and selecting features to determine which features, including the job function and the preferences, contributes to training the ML models.

5. The AI system of claim 4 wherein the performing machine learning (ML) training comprises:
employing decision trees to partition the dataset based on feature values;
creating an ensemble of the decision trees, wherein each ensemble is trained on random data and features to achieve robust predictions through a voting mechanism;
building a series of weak learners by utilizing extreme gradient boosting, wherein the built series of the weak learners are combined for enhanced predictive power; and
performing logistic regression process as a classification algorithm.

6. The AI system of claim 5 wherein evaluating and/or testing the trained ML models comprises:
evaluating the trained ML models with the dataset with evaluation metrics including one or more selected from the group consisting of accuracy, precision, recall, F1 score, and area under the receiver operating characteristic (ROC) curve; and
performing hyperparameter tuning, wherein the hyperparameter is tuned either systematically exploring a predefined set of hyperparameters or randomly sampling hyperparameters.

7. The AI system of claim 1 wherein the one or more content providers comprise a source of data, a source of videos, a source of texts, a source of audios, and/or Internet websites.

8. The AI system of claim 1 wherein the one or more system databases comprise lists of devices and products with features and manufacturers information.

9. The AI system of claim 1 wherein the guidance server accesses one or more AI sources comprise a listing of products and clinical applications sources to determine the best match for the searcher query, and accesses an artificial intelligence pattern list including a listing of patterns determined to indicate the best match to determine that the pattern of the content matches at least one of the patterns matches the searcher query, and to generate an output to indicate a variety of options that match the researcher query.

10. The AI system of claim 1 wherein the guidance server is connected to one or more treatment devices/products guideline servers that offers different treatment devices and/or product recommendations.

11. The AI system of claim 1 wherein the operations further comprise generating an output to indicate a percentage of the content that is artificial intelligence content.

12. A method to deliver artificial intelligent (AI)-generated recommendations and guidance for selections of clinical devices and/or products via either (i) an AI system including a guidance server or (ii) an integrated development environment (IDE) system in a cloud system, comprising:
receiving a user input including a job function, preferences and requirements;
receiving a dataset corresponding to the job function from one or more system database, wherein the dataset comprises lists of devices and products with features and manufactures information;
performing machine learning (ML) training by using the dataset with diverse ML models;
evaluating and/or testing the trained ML models by using the dataset, wherein the evaluating and/or testing the trained ML models comprises selecting the best ML model that demonstrates superior performance among the ML models;
generating a prediction of recommended products and/or devices, based on the best ML model;
performing matching process of the recommended products and/or devices with products/devices contents in a content provider; and
outputting the information of the matched products/devices contents of the content provider.

13. The method of claim 12 further comprising splitting the dataset corresponding to the job function into a training dataset and a testing dataset, wherein the ML training is performed by using the training dataset and the testing the trained ML models is conducted with the testing dataset.

14. The method of claim 12 further comprising performing k-fold cross-validation in which the dataset is divided into k subsets and the ML models undergo training and testing k times for robust evaluation, where k is an integer.

15. The method of claim 12 further comprising identifying and selecting features to determine which features, including the job function and the preferences, contributes to training the ML models.

16. The method of claim 15 wherein the performing machine learning (ML) training comprises:
employing decision trees to partition the dataset based on feature values;
creating an ensemble of the decision trees, wherein each ensemble is trained on random data and features to achieve robust predictions through a voting mechanism;
building a series of weak learners by utilizing extreme gradient boosting, wherein the built series of the weak learners are combined for enhanced predictive power; and
performing logistic regression process as a classification algorithm.

17. The method of claim 16 wherein evaluating and/or testing the trained ML models comprises:
evaluating the trained ML models with the dataset with evaluation metrics including one or more selected from the group consisting of accuracy, precision, recall, F1 score, and area under the receiver operating characteristic (ROC) curve; and
performing hyperparameter tuning, wherein the hyperparameter is tuned either systematically exploring a predefined set of hyperparameters or randomly sampling hyperparameters.

18. The method of claim 12 wherein the one or more content providers comprise a source of data, a source of videos, a source of texts, a source of audios, and/or Internet websites.

19. The method of claim 12 wherein the one or more system databases comprise lists of devices and products with features and manufacturers information.

20. The method of claim 12 further comprise generating an output to indicate a percentage of the content that is artificial intelligence content.
